(19) United States
(12) Patent Application Publication
(10) Pub. No.: US 2016/0296401 A1
(43) Pub. Date: Oct. 13, 2016

(54) STRAP CLAMP ASSEMBLY

(71) Applicant: INNOVATIVE MEDICAL PRODUCTS, INC., Plainville, CT (US)

(72) Inventors: Earl COLE, Prospect, CT (US); Tim BLACKWELL, Jupiter, FL (US)

(73) Assignee: INNOVATIVE MEDICAL PRODUCTS, INC., Plainville, CT (US)

(21) Appl. No.: 15/186,451

(22) Filed: Jun. 18, 2016

Related U.S. Application Data
(63) Continuation-in-part of application No. 13/999,289, filed on Jan. 22, 2014.
Application No. 13/999,289 is a continuation-in-part of application No. 15/050,290, filed on Feb. 22, 2016.

(51) Int. Cl. A61G 13/10; A61F 5/37

(52) U.S. Cl. CPC A61G 13/101; A61F 5/3776

(57) ABSTRACT

A clamping device configured to secure a strap to a rail of a support and/or operating table so as to preventing the patient from slipping, providing optimal patient stability when the operating table is placed in angle positions, and eliminating patient re-positioning during surgical procedure. The clamping device comprising a body portion with a rail channel with a rail flange located on upper arms so as to engage the upper surface of the side rail and a pivot jaw assembly with jaw rail flange portions is connected to sides of the body portion hingedly operated between an open and closed position. The clamping device further comprising a body portion with a strap channel assembly having strap flange portions configured to hold a strap in the strap channel assembly and to secure the strap a side rail using a clamp bar and the rail flanges of the clamp assembly to a clamp to the side rail of an operating table.

### CROSS-REFERENCE TO RELATED APPLICATION

This application is a continuation-in-part and claims the benefit of U.S. patent application Ser. No. 13/999,289, filed Jan. 22, 2014, entitled “Operating table patient support pad clamp,” and also is a continuation and claims the benefit of U.S. patent application Ser. No. 15/050,290, filed Feb. 22, 2016, entitled “Pad Assembly, System, Method Of Pre-Load Positioning Of Patient For Medical Procedure And Kit,” which are incorporated by reference in their entirely.

### FIELD OF THE INVENTION

The present invention relates to patient positioning systems for use in surgical procedures and, more particularly, to a clamping device that secures a strap to a rail of a support and/or operating table preventing the patient from slipping, providing optimal patient stability when the operating table is placed in angle positions, and eliminating patient re-positioning during surgical procedure.

### BACKGROUND OF THE INVENTION

In the prior art, many of the positioning devices use straps secured to the side rails of the operating table to prevent the patient from slipping during surgical procedures. In some instances the straps are attached to a patient positioning pad that may be formed of various materials, e.g. foam, high density open cell foam materials, gel, beanbag, shoulder restraints, braces, and tape. Some of the shortcomings of these prior art positioning devices are that they do not meet patient positioning demands of new advanced robotic surgical procedures that require holding a patient in a desired position in any angle for a desired surgical procedure, e.g. front-to-back, back-to-front, or side-to-side. Moreover, these prior art positioning devices may allow the patient to slip when placed in steep Trendelenburg positions that requires re-positioning during surgical procedures, e.g. patient slippage occurs with heavier and larger sized patients are placed in steep Trendelenburg positions.

Patient slippage on the operating table is frequently encountered when a steep angles are employed due to gravity, reduced friction, and/or fluids generated or involved in a surgical procedure. Modern robotic surgical procedures, for example, position the operating room table in various angles (e.g. Trendelenburg, reverse-Trendelenburg or otherwise side-to-side angles) to move organs away from the site of the surgical procedure and may use a patient positioning pad system. Even with a patient positioning pad system, the slippage problem still occurs in surgical procedures when larger, heavier patients when the operating table is positioned to have the patient's head-elevated, to have the patient's feet-elevated, and/or to elevate from side-to-side. It is undesirable for the patient to slip, shift or otherwise move on the operating table during the surgical procedure especially with an increase in modern, endoscopic surgical procedures using robotic tools as this may cause tissue and nerve damage as well as the delay due to re-positioning. In robotic assisted surgical procedures, slipping and shifting of the patient on the operating table causes movement of the site of the surgical procedure. Numerous problems are caused when the site the site of the surgical procedure shifts including, for example, trauma, tearing at the site, or other tissue and nerve injuries to the patient that may cause adverse events and prolong the recovery of the patient. As a result, there is a long-felt need to eliminate the patient from shifting or otherwise changing position on the operating table during a surgical procedure.

Some of the shortcomings of the prior art clamping devices are that conventional rail clamps are not useful to secure operating table straps or the straps of a patient positioning pad system as the straps prevent a rail clamp from joining and/or sliding along the rail. Patient positioning pad systems rely on looping straps around the side rail of the operating table so as to attach positioning pad to the operating table. Some practices place additional operating table straps over the chest and limbs of the patient, whereby these prior art methods can cause unnecessary pressure on the neck, shoulders or arms, resulting in, for example, nerve damage and pressure ulcers. As a result, a need exists for a clamping device for operating table configured to join a strap to the rail of the operating table at any point between the ends of the rail.

Some of the shortcomings of conventional rail clamping devices include requiring placement at an exact location to attach to a side rail of the operating table. Conventional rail clamps are designed to join at a rail post by positioning the rail clamp slot adjacent the rail post, allowing clearance over the rail, or by sliding from an end of the rail to the desired location. Conventional rail clamps are prevented from joining at the rail post when straps are used because (1) the strap secured around the rail prevents positioning the rail clamp slot adjacent the rail post, and (2) the strap prevents the sliding downward and rotational movement necessary to attach the rail to the operating table. As a result, conventional rail clamps are prevented from joining the rail any point between the ends of the rail. A need exists for a clamping device configured to join at any point between the ends of the rail when using straps and or straps of a patient positioning pad system.

### SUMMARY OF THE INVENTION

It is an object of the present invention to provide a rail clamp assembly to secure the straps of a patient positioning pad firmly to the side rail of an operating table during a surgical procedure so as to eliminate the patient from shifting on operating table or otherwise changing position on the patient positioning pad.

It is an object of the present invention to provide a rail clamp assembly to join firmly to the side rail of an operating table at any point along the side rail during a surgical procedure when patient positioning pads are used.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Description of the Embodiments, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 is a schematic, bottom perspective view of a clamp assembly in accordance with an embodiment of the present invention;

FIGS. 2A, 2B and 2C illustrate rail clamps of the prior art wherein FIG. 2A is a schematic perspective view illustrating a rail clamp of the prior art, FIG. 2B is a schematic, bottom view illustrating the arm and body of a rail clamp of the prior art, and FIG. 2C is an expanded perspective view illustrating a rail clamp connecting to the side rail over a rail post of to the prior art;

FIG. 3 is a schematic, perspective top view of a clamp assembly in accordance with an embodiment of the present invention;

FIG. 4 is a perspective side view illustrating the body of a clamp assembly in accordance with an embodiment of the present invention;

FIG. 5 is a perspective bottom view illustrating the body of a clamp assembly according to the invention;

FIG. 6 is a perspective top view illustrating a lever of a clamp assembly in accordance with an embodiment of the present invention;

FIG. 7 is a side view illustrating a threaded swivel screw of a clamp assembly in accordance with an embodiment of the present invention;

FIGS. 8A and 8B are a cross-sectional, side view, taken along lines A-A of FIG. 3, illustrating a clamp assembly clamping a strap to the side rail of an operating table in accordance with an embodiment of the present invention

FIG. 9 is a schematic perspective expanded top view of a clamp assembly in accordance with an embodiment of the present invention;

FIG. 10 is a schematic perspective view of a clamp assembly clamping a strap to the side rail of an operating table in accordance with an embodiment of the present invention;

FIG. 11 is a side, perspective view of the clamp in the environment of operably connecting a clamp assembly and pivot jaw assembly in the open position to a side rail of an operating table in accordance with an embodiment of the present invention; and

FIG. 12 is a side, perspective view of the clamp in the environment of operably connecting a clamp assembly and pivot jaw assembly in the closed position to a side rail of an operating table in accordance with an embodiment of the present invention.

### DESCRIPTION OF THE EMBODIMENTS

Non-limiting embodiments of the present invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

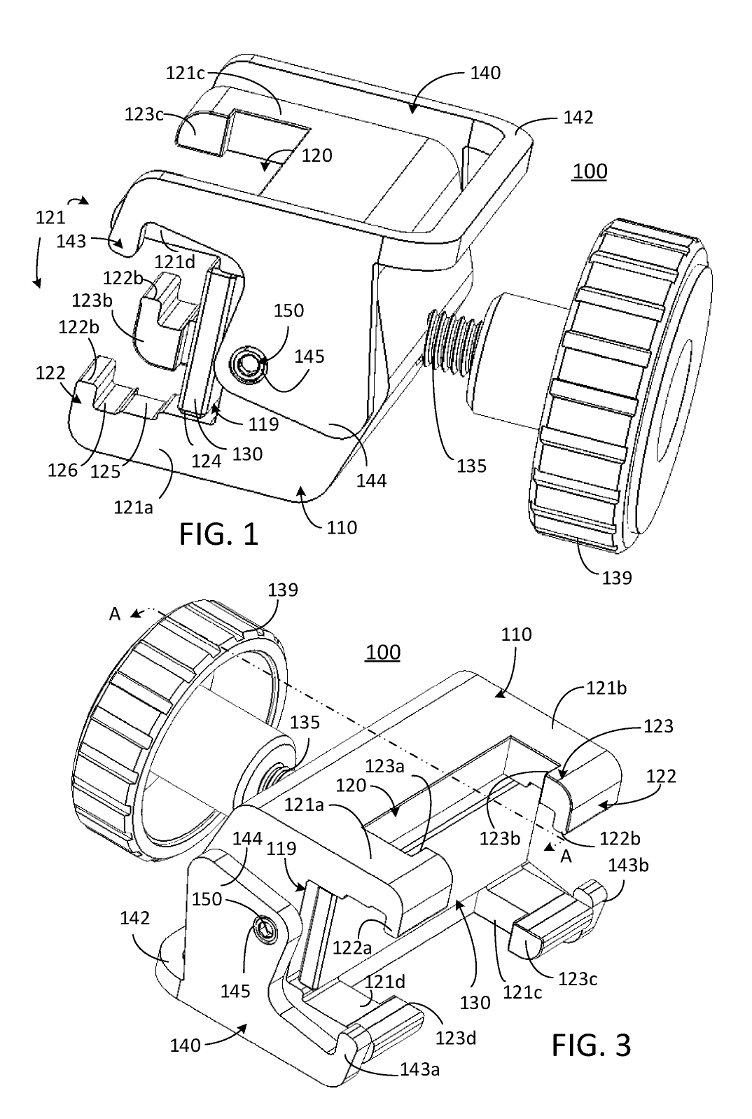

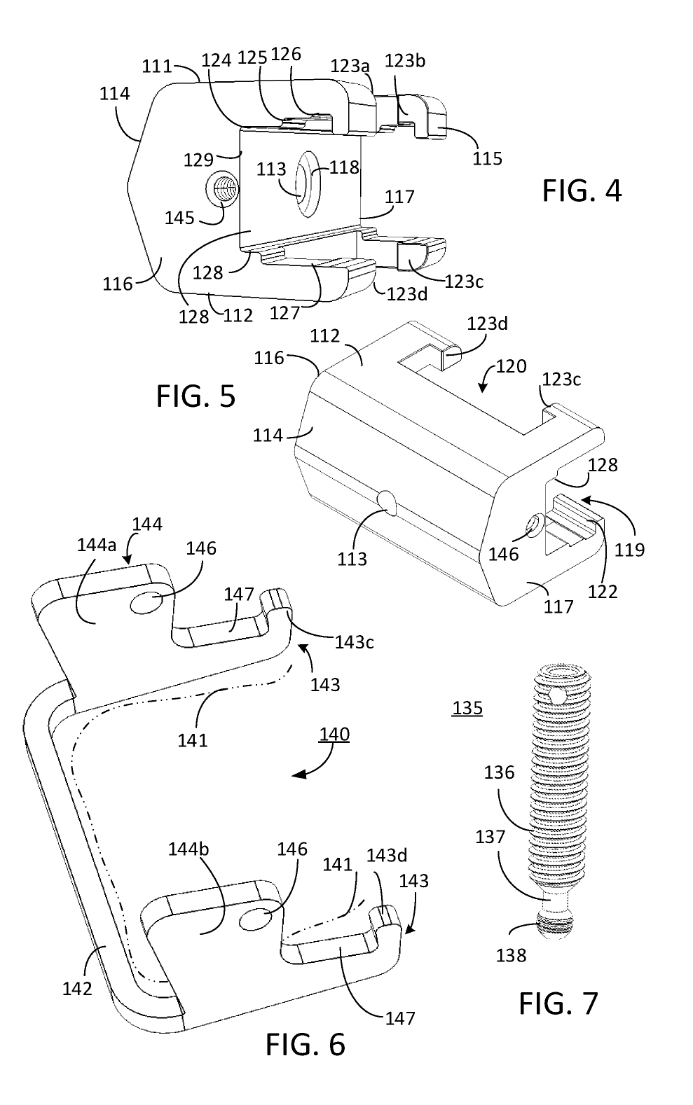

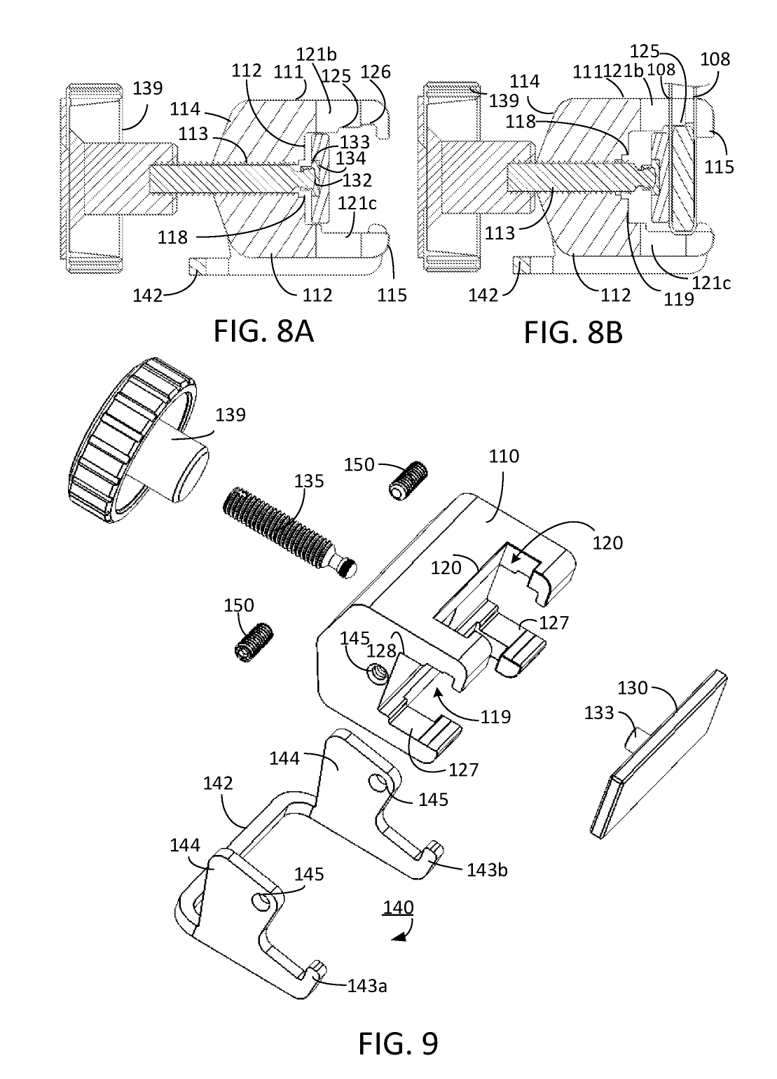

As is illustrated in FIGS. 1, and 3-12, the present invention relates to a clamp assembly 100 for securing to a rail 102 of a support table and/or operating table 104, sometimes called an OR table, as is illustrated in FIGS. 8A-8B, 10 and 11-12. According to an embodiment of the present invention, the clamp assembly 100 is described in the environment of a surgical procedure where the operating table 104 is angled with the patient secured by a strap 106 to the rail 102 of the operating table 104. Similarly, the clamp assembly 100 can clamp to one or more straps 106 of a patient positioning pad system 108 and such patient positioning pad systems 108 are generally available in the marketplace, for example, from Innovative Medical Products, Inc. of Plainville, Conn. sold under the product name TrenMax™ and Xodus Medical Inc. Pittsburgh, Pa. as shown U.S. Pat. Nos. 8,464,720 and 8,511,314. It should be appreciated by one skilled in the art the clamp assembly 100 may be useful for clamping individual straps 106, items and things to a rail 102 of thee support and/or operating table 104. According to the advantages of the present invention, the clamp assembly 100 clamping a strap 106 to the rail avoids slippage problems of conventional straps and/or patient positioning pad systems so as to reduce trauma that may be caused by slippage to patients positioned at any angle on the operating table 104 during a surgical procedure including Trendelenburg, reverse-Trendelenburg, and at side-to-side angles.

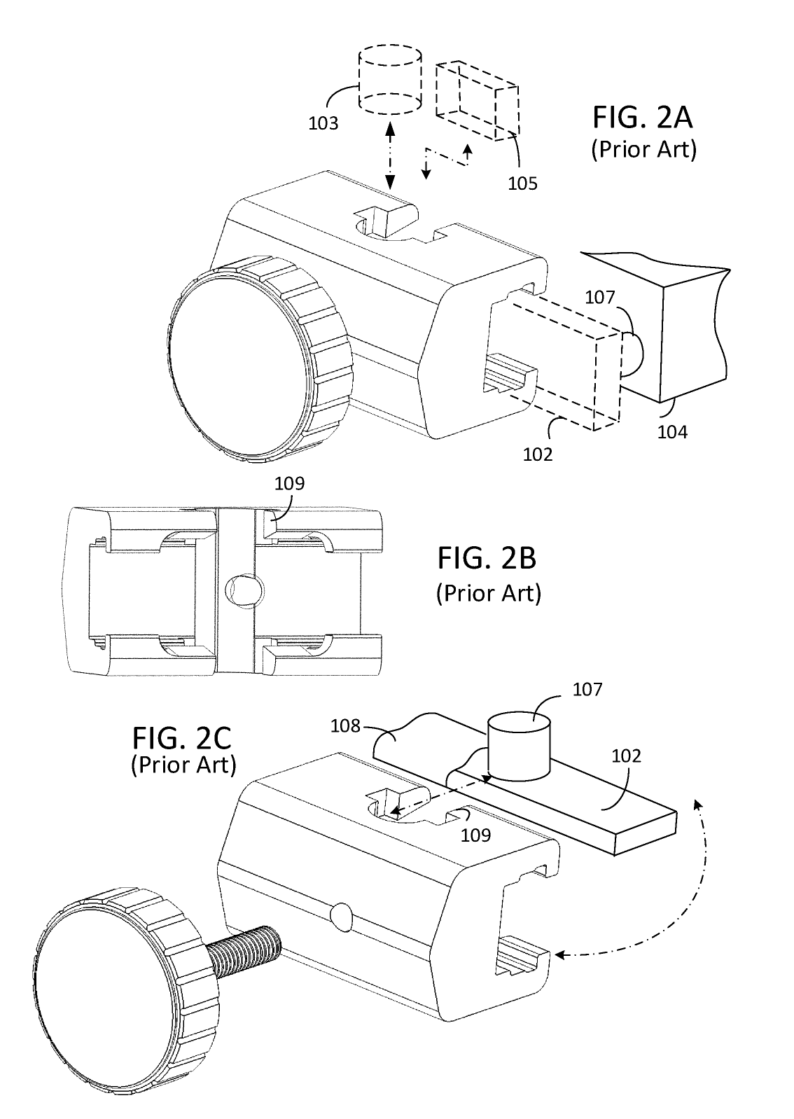

According to the present invention, as illustrated in is shown in FIGS. 2A-2C, prior art rail clamps for retractors and other items and objects are unable to be used to secure straps 106 to the side rail 102 of the operating table 104. Conventional rail clamping devices are used during surgical procedures that are typically secured to a side rail 102 attached at a rail post 107 to the side of the operating table 104. These prior art rail clamping devices utilize a construction with a clamp member and a set screw, whereby the set screw engages the side rail which secures the clamping member to the side rail 102, whereby a round post 103 or a square post 105 of a surgical attachment, e.g. retractor, limb holder, post or other item is secured thereto, as is shown in FIG. 2A. The set screw and knob of conventional rail clamps is adapted to tighten through a central hole, as is shown in FIG. 2B, allowing a knob and a threaded post to tighten against the round post 103, or square post 105, to the rail 102, or, alternatively, the set screw also may tighten directly to the rail 102. As is illustrated in FIG. 2C, conventional rail clamps can only be joined between the ends of the rail 102 at a rail post 107, whereby a rail clamp slot 109 is positioning adjacent the rail post 107 attaching the rail 102 to the operating table 104 with a sliding downward movement and then rotating the clamp body 90 degrees (90°). A strap 106 of a patient positioning system 108 prevents conventional rail clamping devices from joining at the rail post 107 as well as at any point between the ends of the rail because (1) a strap of the patent positioning pad secured around the rail prevents positioning the rail clamp slot adjacent the rail post, and (2) the strap prevents the sliding downward and rotational movement necessary to attach the rail to the operating table as shown in FIG. 2C. A long-felt need exists for a clamping device to overcome the advantages and a clamping device 100 configured to join at any point between the ends of the rail 102 when using straps 106 and or straps of a patient positioning pad system 108.

As is illustrated in FIGS. 1, and 3-12, according to an embodiment of the present invention a clamp assembly 100 comprises a body portion 110 configured with a rail channel 119 and a strap channel assembly 120, a clamp bar 130 operably coupled to a swivel screw 135 and knob 139, and a pivot jaw assembly 140. The rail channel 119 and strap channel assembly 120 may be formed to provide a plurality of arms 121 extending from the body portion 110 such as, for example, individually identified arms 121a, 121b, 121c, and 121d extending from the corners of the body portion 110. Arms 121a and 121b are utilized as upper arm members to rest the clamp assembly 110 on the upper surface 102a or top of rail 102. The arms 121a and 121b may be configured with a rail flange 122, individually identified as rail flanges 122a and 122b, configured to hang the clamp 100 on the rail 102 of the support table 104. Arms 121c and 121d are utilized as lower arm members slide over on the lower surface 102a or bottom of rail 102 and join the clamp assembly 110 to the rail 102 with a jaw rail flange portion 143 of the pivot jaw assembly 140. In addition, the arms 121a, 121b, 121c, and 121d may further be configured with strap flange 123 that extends inwardly from the arms 121a-121d of the body portion 110 such as, for example, individually identified strap flanges 123a, 123b, 123c, and 123d. The body portion 110 may be formed from suitable materials of sufficient strength and durability such as, for example, extruded metal, stainless steel, 6061 T6 aluminum stock, carbon fiber, or other suitable stock materials. The body portion 110 may be milled using computer numeric controlled manufacturing techniques to form arms 121a, 121b, 121c, and 121d. Similarly, CNC manufacturing may be used to form rail flange 122a and 122b, and strap flanges 123a, 123b, 123c, and 123d.

As illustrated in FIGS. 1, 3, 4, 5, 8A-8B through 12, the body portion 110 is configured with a top portion 111, a bottom portion 112, a front portion 114, a rear portion 115, side portion 116, and side portion 117. The body portion 110 may be configured with a centrally disposed hole 113 connecting the front portion 114 and rear portion 115 and adapted to accept a swivel screw 135 shown in FIG. 7. Centrally disposed hole 113 also may be configured with a counter-sink hole 118 adapted to receive a swivel foot 133 of the clamp bar 130 as is shown in FIG. 8A-8B. The body portion 110 may be configured with a rail channel 119 formed in the rear portion 115 and extending between the side portions 116, 117. A strap channel assembly 120 may be formed in the rear portion 115 of the body portion 110 extending between the top and bottom portions 111, 112.

Referring to FIG. 6, a pivot jaw assembly 140 is configured to connect to the base portion 110 so as to provide attachment to the lower part of the rail 102 of the operating table 104. The pivot jaw assembly 140 comprises a base body portion 141 of a predetermined elongated shape that may be stamped and bent into a U- or C-shape so as to be joined by a set screw 150 to each side portion 116, 117 of the base portion 110. The pivot jaw assembly 140 may be formed from suitable materials of sufficient strength and durability such as, for example, extruded metal, stainless steel, 6061 T6 aluminum stock or other suitable stock materials. The predetermined shape of the pivot jaw assembly 140 comprises a handle portion 142, a jaw rail flange portion 143, individually identified jaw rail flange portions 143a and 143b, and a hinge portion 144 configured with holes 145 for attaching the pivot jaw assembly 140 to the base portion 110. Specifically, the pivot jaw assembly 140 is joined by two set screws 150 via set screw holes 145 in the hinge portion 144 and set screw holes 146 formed in each side portion 116, 117. The jaw rail flange portion 143 operates to engage the rail 102 in a closed position, whereby, individually identified jaw rail flange portions 143a and 143b cooperate with rail flanged portions 122a and 122b when the knob is tightened or loosened moving the clamp bar 130 so as to clamp the strap 106 against rail 102 as illustrated in FIGS. 8A-8B through 12.

Referring to FIGS. 1, 3-5, 8A-8B, 10-12, the rail channel 119 is dimensioned to accept various dimensions of a side rail of the operating table 106, for example, a rail 102 of a standard dimension, and a taller, thinner rail of other standard rail dimensions identified by broken-line element 102c shown in FIGS. 8A and 8B. The rail channel 119 is further configured with a cavity 128 for receiving the clamp bar 130 in the retracted or untightened position, for example, as shown in FIGS. 1 and 3. The cavity 128 is configured with cavity surface 129 that may be formed with the counter sink hole 118 configured to receive the swivel foot 133 so as to allow the clamp bar 120 to retract fully adjacent the cavity surface 129 when attached to the swivel screw 135.

The rail channel 119 may be configured with an upper arm surface 124 on upper arms 121a, 121b so as to rest on an upper edge 102a of the rail 102 as shown in FIGS. 8A-8B and 10-12. The upper surface 124 may be further be formed with a first step 125 and a second step 126 to accommodate different rail 102 dimensions and to improve positioning of the clamp 100 on the upper surface of the rail 102 of the support table 104. For example, the first step surface 125 is useful for registering, aligning the upper edge 102a of the rail 102 so as to abut in adjacent relationship the upper arm surface 124 of arm 121 when tightening the clamp assembly 100. The second step surface 126 is useful for registering upper edge 102a of the rail 102 for a thinner dimensioned rail 102c as well as to provide additional space when rotating and fitting the lower arms 121c, 121d over the lower edge of the rail 102. A lower arm rail surface 127 is dimensioned to slide over the lower edge 102b of the rail 102 and formed to abut adjacent the lower edge 102b when tightening the clamp assembly 100.

As is illustrated in FIG. 7, the swivel screw 135 is configured with an upper screw portion 136, a transition portion 137, and a lower screw portion 138. The upper screw portion 136 may be formed with threads adapted to be received in the centrally disposed hole 113 of the body portion 110. The upper screw portion 136 may further be adapted to be received by the knob 139 and secured to the knob 139 by a post or set screw. The swivel screw 135 may be formed from suitable materials of sufficient strength and durability that may be tooled and threaded such as, for example, extruded metal, stainless steel, 6061 T6 aluminum stock or other suitable stock materials.

Referring to FIGS. 7 and 9, a swivel foot 133 is configured to operably connect to the swivel screw 135 so as to provide the clamping action of the clamp bar 130 against the strap 106 against a rail 102 of the operating table 106 and is illustrated in FIGS. 8A-8B. The clamp bar 130 and swivel foot 133 may be formed from suitable materials of sufficient strength and durability such as, for example, extruded metal, stainless steel, 6061 T6 aluminum stock or other suitable stock materials. The lower screw portion 138 may be formed with threads adapted to be received in the recessed hole 134 of the swivel foot 133 is illustrated in FIGS. 8A-8B. As is illustrated in FIG. 9, the swivel foot 133 may be secured to the clamp bar 130 in the recessed hole 134 by welding or the like. The lower screw portion 138 of the swivel foot 133 may be fastened in the hole 132 by a threaded connection as to provide wide-ranging clamping of straps 106 of various dimensions to the rail 102. Alternatively, the swivel foot 133 may be fastened the clamp bar 130 by crimping so as to allow movement angularly to the extent the swivel foot 130 rotates to contact the transition area 137 in the plane of in three dimensions (e.g. x, y and z coordinates) so as to adjust and provide complete clamping to the straps 106 of various dimensions, thicknesses and materials as disposed in the rail channel 119.

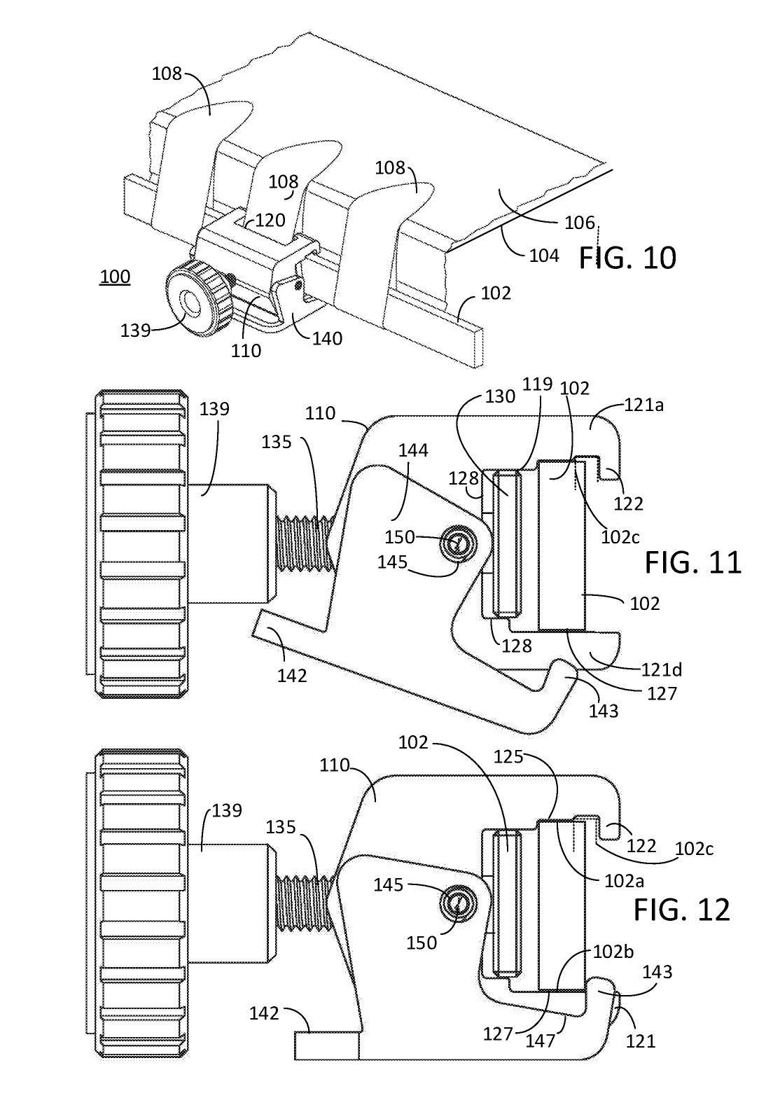

In operation, as is illustrated in FIGS. 8A-8B and 10-12, the clamp assembly 100 is used as to clamp and secure a strap 106 at any point along the rail 102 of an operating table 104. An individual strap 106, as shown in FIG. 8B, and one or more straps 106 of a patient positioning pad 108, as shown in FIG. 10, may be secured around the rail 102 of a support and/or operating table 104. As shown in FIGS. 8B, 10 and 11, the clamp assembly 100 may be positioned on the upper surface 102a of the rail 102 with the strap 108 disposed in the strap channel assembly 120 between the arms 121a, 121b, 121c, 121d and the strap flanges 123a, 123b, 123c and 123d. The rail flanges 122a and 122b are position on the rail 102 resting on the upper surface 102a and the upper arm surface 124 (e.g. the first step 125 and the second step 126). The clamp assembly 100 may be rotated slightly towards the rail 102 passing the lower arms adjacent the lower rail surface 102b with the pivot jaw assembly 130 opened downwardly as is illustrated in FIG. 11. The pivot jaw assembly 130 may be rotated upwardly to place the jaw rail flange portions 143a and 143b adjacent the rear surface of the rail 102 as is illustrated in FIGS. 8B and 12. The clamp bar 130 of the clamp assembly 100 may be tightened and loosened using knob 139 so as to force be swivel screw operably connected to the clamp bar 130 against the strap 106 and rail 102 so as to secure the strap 106 to the rail 102 and the clamp assembly 100 to the rail 102. The force of the clamp bar 130 against the strap 106 and rail 102 operably connects the jaw rail flange portions 143a and 143b of the pivot jaw assembly 140 and the rail jaw rail flange portions 122a and 122b of the upper arms against the inner surface of the rail 102 so as to secure and hold the strap 106 to the rail 102.

Accordingly, the present invention provides a clamp assembly 100 to secure a strap 102 and/or one or more straps 102 of a patient positioning pad system 108 securely to the side rail 102 of an operating table 104 during a surgical procedure so as to eliminate the patient from shifting on operating table 104 or otherwise changing position on the patient positioning pad 108. The over present invention provides numerous advantages over conventional positioning devices and satisfies patient positioning demands present in new advanced robotic surgical procedures that require holding a patient in a desired position in any angle for a desired surgical procedure, e.g. front-to-back, back-to-front or side-to-side. Moreover, the invention overcomes slippage problems of conventional systems having advantages in providing secure clamping of the strap 106 to the rail 102 of the operating table 104 when the patient is placed in steep Trendelenburg positions that requires re-positioning during surgical procedures, e.g. patient slippage occurs with heavier and larger sized patients are placed in steep Trendelenburg positions.

While certain configurations of structures have been illustrated for the purposes of presenting the basic structures of the present invention, one of ordinary skill in the art will appreciate that other variations are possible which would still fall within the scope of the appended claims. Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A device for clamping to the rail of an operating table, the operating table having a side rail, the device comprising:
a body portion comprising a rail channel with one or more arms disposed on opposite sides of the side rail of the operating table, said body portion configured with upper rail arms having a rail flange adapted to hang the device on the upper surface of the side rail, said body portion configured with a strap channel assembly and strap flanges on said one or more arms adapted to receive a strap between the body portion and the side rail;
a clamp bar disposed in said rail channel of said body portion, said clamp bar adapted to apply force to the side rail of the operating table;
a swivel screw mechanism operably connected between said body portion and said clamp bar such that said clamp bar is movable in opposing directions thereby frictionally engaging said clamp bar with the side rail of the operating table; and
a pivot jaw assembly configured with a hinge portion and a jaw rail flange portions, said pivot jaw assembly connected to said body portion at said a hinge portion to allow movement of said pivot jaw assembly at said hinge portion between open and closed positions.

2. The device of claim 1, wherein said a clamp bar further configured with a swivel foot disposed at a mid-portion thereof to operably connect to said swivel screw.

3. The device of claim 1, wherein said clamp bar, rail flange on said upper arm portions and said jaw rail flange portions are configured to hold a strap secured around said side rail between said clamp bar and the side rail of an operating table.
